(19) United States
(12) Patent Application Publication
(10) Pub. No.: US 2026/0345674 A1
(43) Pub. Date: Nov. 5, 2026

(54) PHARMACEUTICAL COMPOSITION

(71) Applicant: Aerotech Australia Pty Ltd., Woolner (AU)

(72) Inventor: Roger Phillip Gordon Leach, Woolner (AU)

(73) Assignee: Aerotech Australia Pty Ltd., Woolner, NT (AU)

(21) Appl. No.: 19/486,649

(22) PCT Filed: May 22, 2024
(86) PCT No.: PCT/AU2024/050528

§ 371 Date: Nov. 21, 2025

(30) Foreign Application Priority Data
May 23, 2023 (AU) 2023901609

(51) Int. Cl. A61K 33/10; A61K 9/00; A61K 31/10; A61K 31/385; A61K 36/752; A61K 47/10; A61K 47/14; A61K 47/34; A61K 47/36; A61P 11/00; A61P 17/00; A61P 31/22

(52) U.S. Cl. CPC A61K 33/10; A61K 9/0014; A61K 9/007; A61K 31/10; A61K 31/385; A61K 36/752; A61K 47/10; A61K 47/14; A61K 47/34; A61K 47/36; A61P 11/00; A61P 17/00; A61P 31/22

(57) ABSTRACT

Provided herein is a pharmaceutical composition of a magnesium salt with alpha lipoic acid and/or methylsulfonylmethane (MSM), useful for the treatment, prevention or reduction in severity of viral infections and/or skin conditions. Methods for preparing the pharmaceutical composition, and methods of using the pharmaceutical composition in therapy are also provided.

### CROSS-REFERENCE TO RELATED APPLICATIONS

This application is a U.S. National Phase Patent Application based on International Patent Application No. PCT/AU2024/050528, filed on May 22, 2024, which claims priority to Australian Patent Application No. 2023901609, filed on May 23, 2023, the contents of both of which are incorporated by reference herein in their entirety as if fully set forth herein.

### FIELD

The present invention relates to a pharmaceutical composition, in particular a pharmaceutical composition useful for the treatment or prevention of viral infections and/or skin conditions, for example such as herpes zoster infection, varicella-zoster infection, chicken pox, shingles, hidradenitis suppurativa, psoriasis, acne, atopic dermatitis, atopic eczema, postherpetic neuralgia, pulmonary fibrosis and SARS-Cov-2.

### BACKGROUND

Acne, or acne vulgaris, is a disorder of the pilosebaceous follicles found in the face and upper trunk. At puberty, androgens increase the production of sebum from enlarged sebaceous glands that become blocked. Propionibacterium acnes is typically involved in lesion production, although its exact role is unclear. Comedones (follicles impacted and distended by incompletely desquamated keratinocytes and sebum) may be open (blackheads) or closed (whiteheads). Inflammation leads to papules, pustules and nodules.

Almost every teenager can expect to experience acne to some degree during the adolescent years, although it is usually mild. Moderate to severe acne affects about 20% of young people and can cause severe psychological problems, undermining self-assurance and self-esteem. While there are various treatments available for acne, these have varying degrees of success and have side effects.

Hidradenitis suppurativa is a long-term (chronic) skin condition. The cause is unknown and the disease only affects areas of skin containing apocrine sweat glands. Inflammation of the apocrine sweat gland-bearing areas leads to painful and repeated lumps of pus (boils or abscesses). Suppuration means formation or discharge of pus. The areas, commonly the armpits and groin, leak pus and are difficult to heal. Eventually, scarring occurs. Other areas such as under the breasts and on the vulva, the scrotum, the buttocks and the skin in front of the anus (the perineum) are sometimes affected.

The wounds caused by the boils and abscesses heal poorly, leaving scars. In severe cases, the pus tunnels down under the skin surface. The tunnels formed are called sinus tracts. Multiple areas of hidradenitis can become linked under the skin surface by a network of interconnected sinus tracts. As a consequence, the inflammation, and sometimes infection, can travel deeper and become more widespread. The eventually healed areas are full of thick scar tissue, and the scarring left behind can be as unsightly as the discharging wounds.

Hidradenitis suppurativa (HS) is difficult to control with medical treatment, with the aim being to catch the disease in its early stages and to treat and control milder forms of disease. Treatments include the use of topical antibiotics, short courses of antibiotic tablets, prolonged courses of antibiotics, retinoids, steroids and immune system modulators such as adalimumab. Chronic HS often requires surgery in the form of incision and drainage, wide-scale removal (excision) of affected areas, and carbon dioxide laser treatment. None of these treatments are curative and may result in significant scarring that requires skin grafts and other plastic surgery techniques.

HS can prevent normal working and social activities. Psychological problems are common, as are difficulties in sexual relationships. These problems can either be directly due to the pain and messiness of the condition or due to embarrassment and body image problems.

Psoriasis is a chronic inflammatory skin disease that manifests as erythematous, dry, scaling plaques resulting from hyperkeratosis. The plaques are most often found on the elbows, knees and scalp, though more extensive lesions may appear on other parts of the body, notably the lumbosacral region. The most common treatment of mild to moderate psoriasis involves topical application of a composition containing a corticosteroid as the active ingredient. While efficacious, application of corticosteroids has the disadvantage of a number of adverse effects such as skin atrophy, striae, acneiform eruptions, perioral dermatitis, overgrowth of skin fungus and bacteria, hypopigmentation of pigmented skin and rosacea.

Herpes zoster, also known as shingles is a condition which can cause a painful rash, redness, and/or lesions such as fluid-filled blisters on the skin. Symptoms can also include itching, tingling, or a burning sensation in the area affected, as well a fever, chills, headache and upset stomach. Postherpetic neuralgia is a common complication of shingles, with the condition affecting the skin and/or nerve fibres, often causing pain which persists after other symptoms have diminished. Herpes zoster is caused by the varicella zoster virus, which is also responsible for other conditions such as chickenpox. Herpes zoster ophthalmicus is a subset of herpes zoster infection that affects the ophthalmic division (V1) of the trigeminal cranial nerve, which can cause further complications such as ocular disease, neuropathy and/or migraine. Whilst there are some therapies including antiviral agents such as acyclovir, famcicovir and valacyclovir, there remains a need for further treatments for conditions such as herpes zoster, and related disorders.

Atopic dermatitis/eczema is a condition characterised by itchiness and/or redness of the skin. In many cases the condition tends to flare periodically. Symptoms can include dry skin, itching, red to brown-gray patches, raised skin, and thickened, cracked, swollen or raw skin. Atopic dermatitis/eczema can be associated with conditions such as viral infections. For example, viruses and other infective agents can readily infect the skin of some patients having atopic dermatitis, aggravating the condition.

It would be desirable to provide a therapy for treating viral infections and/or skin conditions, for example such as herpes zoster infection, varicella-zoster infection, herpes simplex infection, chicken pox, shingles, cold sores, genital herpes, hidradenitis suppurativa, psoriasis, acne, atopic dermatitis, atopic eczema, postherpetic neuralgia, pulmonary fibrosis and SARS-CoV-2.

### SUMMARY

The present inventor has identified a pharmaceutical composition with unexpected efficacy in treating a range of viral infection-associated and/or skin conditions.

Accordingly, there is provided a pharmaceutical composition comprising (a) a magnesium salt; and (b) lipoic acid and/or methylsulfonylmethane (MSM).

In some embodiments, the magnesium salt is selected from the group consisting of magnesium chloride and magnesium sulphate.

In some embodiments, the composition is formulated for topical administration.

In some embodiments, the composition comprises magnesium chloride, alpha-lipoic acid and methylsulfonylmethane (MSM).

In some embodiments, the composition further comprises sodium bicarbonate.

In some embodiments, the composition comprises an emollient, humectant, emulsifier, or preservative, or mixtures thereof.

In some embodiments:
i) the emollient is selected from the group consisting of glyceryl stearate, ceteareth-20, ceteareth-12, cetearyl alcohol, cetyl palmitate, dimethicone, cyclopentasiloxane, cyclohexasiloxane, and caprylic/capric triglyceride, or a mixture thereof,
ii) the humectant is selected from the group consisting of glycerol, glycerin, salicylic acid, and sorbitol, or a mixture thereof;
ii) the emulsifier is selected from the group consisting of glyceryl stearate, ceteareth-20, ceteareth-12, cetearyl alcohol, and cetyl palmitate, or a mixture thereof, and
iii) the preservative is selected from the group consisting of phenoxyethanol, ethylhexylglycerin, and sodium benzoate, or a mixture thereof.

In some embodiments, the composition is in the form of a liquid, an aqueous solution, a gel, a suspension, an emulsion, an ointment or a cream.

In some embodiments, the composition comprises by weight of the composition: 10%-65% magnesium sulphate, 5%-20% MSM, 5%-35% magnesium chloride, 1%-10% sodium bicarbonate and/or 0.1%-1% alpha-lipoic acid.

In some embodiments, the composition comprises 20%-50% by weight of the composition of magnesium chloride, alpha-lipoic acid, sodium bicarbonate, magnesium sulphate, and methylsulfonylmethane (MSM).

In some embodiments, the composition comprises lemon extract.

In some embodiments, the composition comprises 50%-80% by weight of lemon extract.

There is also provided a method of treating, preventing, or reducing the severity of a viral infection, or of a disease or disorder associated with a viral infection, or of a symptom of a viral infection, or of a symptom of a disease or disorder associated with a viral infection, in a subject, comprising administering an effective amount of a pharmaceutical composition as disclosed herein to the subject.

In some embodiments, the viral infection, or disease or disorder associated with a viral infection, is selected from the group consisting of herpes zoster infection, varicella-zoster infection, herpes simplex infection, chicken pox, shingles, cold sores, genital herpes, hidradenitis suppurativa, psoriasis, acne, atopic dermatitis, atopic eczema, postherpetic neuralgia and SARS-CoV-2.

In some embodiments, the symptom is selected from the group consisting of cracked skin, skin bleeding, skin peeling, skin redness and pain.

There is also provided a method of treating, preventing, or reducing the severity of a disease or disorder, or of a symptom of a disease or disorder, wherein the disease or disorder is selected from the group consisting of herpes zoster infection, varicella-zoster infection, herpes simplex infection, chicken pox, shingles, cold sores, genital herpes, hidradenitis suppurativa, psoriasis, acne, atopic dermatitis, atopic eczema, postherpetic neuralgia, pulmonary fibrosis and SARS-CoV-2 in a subject, comprising administering an effective amount of a pharmaceutical composition as disclosed herein to the subject.

In some embodiments, the composition is administered topically to the subject.

In some embodiments, the composition is applied to the skin of the subject once daily twice daily or three times daily.

In some embodiments, the topical composition is applied once per day.

In some embodiments, the composition is applied as a spray, cream or liquid.

In some embodiments, the composition is administered to the subject by inhalation.

There is also provided use of a pharmaceutical composition as disclosed herein for the manufacture of a medicament for treating, preventing, or reducing the severity of a viral infection, or of a disease or disorder associated with a viral infection, or of a symptom of a viral infection, or of a symptom of a disease or disorder associated with a viral infection.

In some embodiments, the viral infection, or disease or disorder associated with a viral infection, is selected from the group consisting of herpes zoster infection, varicella-zoster infection, chicken pox, shingles, hidradenitis suppurativa, psoriasis, acne, atopic dermatitis, atopic eczema and postherpetic neuralgia.

In some embodiments, the symptom is selected from the group consisting of cracked skin, skin bleeding, skin peeling, skin redness and pain.

There is also provided use of a pharmaceutical composition as disclosed herein for the manufacture of a medicament for treating, preventing, or reducing the severity of a disease or disorder, or of a symptom of a disease or disorder, wherein the disease or disorder is selected from the group consisting of herpes zoster infection, varicella-zoster infection, herpes simplex infection, chicken pox, shingles, cold sores, genital herpes, hidradenitis suppurativa, psoriasis, acne, atopic dermatitis, atopic eczema, postherpetic neuralgia, pulmonary fibrosis and SARS-CoV-2.

In some embodiments, the composition is administered topically.

In some embodiments, the composition is applied to the skin of a subject once daily, twice daily or three times daily.

In some embodiments, the topical composition is applied once per day.

In some embodiments, the composition is applied as a spray, cream or liquid.

In some embodiments, the composition is administered to the subject by inhalation.

There is also provided a pharmaceutical composition as disclosed herein, for use in treating, preventing, or reducing the severity of a viral infection, or of a disease or disorder associated with a viral infection, or of a symptom of a viral infection, or of a symptom of a disease or disorder associated with a viral infection.

In some embodiments, the viral infection, or disease or disorder associated with a viral infection, is selected from the group consisting of herpes zoster infection, varicella-zoster infection, herpes simplex infection, chicken pox, shingles, cold sores, genital herpes, hidradenitis suppurativa, psoriasis, acne, atopic dermatitis, atopic eczema, postherpetic neuralgia and SARS-CoV-2.

In some embodiments, the symptom is selected from the group consisting of cracked skin, skin bleeding, skin peeling, skin redness and pain.

There is also provided a pharmaceutical composition as disclosed herein for use in treating, preventing, or reducing the severity of a disease or disorder, or of a symptom of a disease or disorder, wherein the disease or disorder is selected from the group consisting of herpes zoster infection, varicella-zoster infection, chicken pox, shingles, hidradenitis suppurativa, psoriasis, acne, atopic dermatitis, atopic eczema, pulmonary fibrosis and postherpetic neuralgia.

In some embodiments, the composition is administered topically.

In some embodiments, the composition is applied to the skin of a subject once daily, twice daily or three times daily.

In some embodiments, the topical composition is applied once per day.

In some embodiments, the composition is applied as a spray, cream or liquid.

In some embodiments, the composition is administered to the subject by inhalation.

There is also provided a pharmaceutical composition as disclosed herein when used in treating, preventing, or reducing the severity of a viral infection, or of a disease or disorder associated with a viral infection, or of a symptom of a viral infection, or of a symptom of a disease or disorder associated with a viral infection.

In some embodiments, the viral infection, or disease or disorder associated with a viral infection, is selected from the group consisting of herpes zoster infection, varicella-zoster infection, herpes simplex infection, chicken pox, shingles, cold sores, genital herpes, hidradenitis suppurativa, psoriasis, acne, atopic dermatitis, atopic eczema, postherpetic neuralgia and SARS-CoV-2.

In some embodiments, the symptom is selected from the group consisting of cracked skin, skin bleeding, skin peeling, skin redness and pain.

There is also provided a pharmaceutical composition as disclosed herein when used in treating, preventing, or reducing the severity of a disease or disorder, or of a symptom of a disease or disorder, wherein the disease or disorder is selected from the group consisting of herpes zoster infection, varicella-zoster infection, herpes simplex infection, chicken pox, shingles, cold sores, genital herpes, hidradenitis suppurativa, psoriasis, acne, atopic dermatitis, atopic eczema, postherpetic neuralgia, pulmonary fibrosis and SARS-CoV-2.

In some embodiments, the composition is administered topically.

In some embodiments, the composition is applied to the skin of a subject once daily twice daily or three times daily.

In some embodiments, the topical composition is applied once per day.

In some embodiments, the composition is applied as a spray, cream or liquid.

In some embodiments, the composition is administered to the subject by inhalation. There is also provided a method of making a pharmaceutical composition as disclosed herein, the method comprising:
combining a magnesium salt with methysulfonylmethane (MSM) and/or alpha-lipoic acid to form a mixture; and
combining the mixture with water or an aqueous solution.

In some embodiments, the method comprises:
(i) combining magnesium sulphate, methylsulfonylmethane (MSM), alpha-lipoic acid and sodium bicarbonate,
(ii) adding magnesium chloride to the product of step (i) to form a mixture,
(iii) combining the mixture formed in step (ii) with water or an aqueous solution.
   In some embodiments, the method comprises a step (iv) in which the aqueous mixture of step (iii) is then heated to about 60 °C to about 90 °C and mixed until homogenous with one or more emulsifiers and oil-phase components.
   In some embodiments, the mixture formed in step (iv) comprises by weight of the composition: 10%-65% magnesium sulphate, 5%-20% MSM, 5%-35% magnesium chloride, 1%-10% sodium bicarbonate and 0.1%-1% alpha-lipoic acid.

In some embodiments, the pharmaceutical composition comprises 20%-50% by weight of the composition of magnesium chloride, alpha-lipoic acid, sodium bicarbonate, magnesium sulphate, and methylsulfonylmethane (MSM), and 50%-80% by weight of water or an aqueous solution.

In some embodiments, an aqueous solution is added, and the aqueous solution comprises lemon extract.

In some embodiments, the lemon extract is lemon juice.

### BRIEF DESCRIPTION OF THE FIGURES

FIG. 1. Treatment of Hidradenitis Suppurativa patient with topical composition. A. Photograph of the patient's face prior to treatment with the topical composition, showing signs of stage 1 HS. B. Photograph taken after three weeks of applying the topical composition to affected skin, with a reduction in visual signs of HS.

FIG. 2. Treatment of shingles (herpes zoster) patient with topical composition. A, B. Photographs of the patient's leg prior to treatment with the topical composition, showing signs of shingles. C₂, D. Photographs taken after three days of applying the topical composition to affected skin, with a reduction in visual signs of shingles.

FIG. 3. Treatment of shingles patient with topical composition. A. Photograph of the patient's neck at the time of initial treatment with the topical composition, showing signs of shingles. B. Photographs taken after applying the topical composition to affected skin, with a reduction in visual signs of shingles.

FIG. 4. Treatment of atopic dermatitis patient with topical composition. A, B. Photographs of the patient's hand shortly after treatment with the topical composition.

### DETAILED DESCRIPTION

### General Techniques and Definitions

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Unless specifically defined otherwise, all technical and scientific terms used herein shall be taken to have the same meaning as commonly understood by one of ordinary skill in the art (e.g., in molecular genetics, biochemistry, and immunology).

Concentrations, amounts, solubilities, and other numerical data may be presented herein in a range format. It is to be understood that such range format is used merely for convenience and brevity and should be interpreted flexibly to include not only the numerical values explicitly recited as the limit of the range, but also to include all the individual numerical values or sub-ranges encompassed within that range as if each numerical value and sub-range is explicitly recited. All numbers expressing quantities, percentages or proportions, and other numerical values used in the specification, are to be understood as being modified in all instances by the term “about”.

“Topical compositions,” as described herein, are compositions, including pharmaceutical compositions, that are suitable for application to skin. “Topical compositions” may be advantageous in that they avoid first-pass metabolism, circumvent gastrointestinal absorption, can allow delivery of an active ingredient with a relatively short biological half-life and/or a narrow therapeutic window, and can allow delivery of an active ingredient to a localized spot on the skin.

As used throughout, by a “subject” is meant an animal or human. Thus, in one aspect, the subject is a mammal such as a primate or a human, including a human patient.

The terms “treat”, “treated” or “treating” as used herein refers to therapeutic treatment and/or prophylactic or preventative measures, wherein the object is to prevent, reduce, eliminate or slow down (lessen) an undesired physiological condition, disorder or disease, or to obtain beneficial or desired clinical results (e.g. decrease signs or symptoms of acne, hidradenitis suppurativa or psoriasis). For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of unwanted side effects.

The term “preventing” as used herein refers to protecting a subject from developing at least one symptom of acne, hydradenitis suppurativa or psoriasis, or reducing the severity of a symptom of acne, hydradenitis suppurativa or psoriasis.

The terms “apply”, “applying” and/or “application”, as used herein in reference to a composition refers to contacting a mammalian, preferably human patient, skin surface with a topical composition.

As used herein, the term “pharmaceutically acceptable” and grammatical variations thereof, as they refer to carriers, diluents, excipients, and reagents or other ingredients of the composition, represent that the materials used in the final composition are not irritating or otherwise harmful to the patient in general and to the skin, in particular, and preferably are well tolerated.

The term “dermatologically acceptable” as used herein refers to a composition or component thereof that may be used in contact with mammalian skin tissue without undue toxicity, incompatibility, instability, allergic response, and the like. The term “dermatologically acceptable carrier” as used herein refers to a carrier that is suitable for topical application to keratinous tissue, has acceptable aesthetic properties, is compatible with the active compounds, and does not cause any safety or toxicity concerns and/or is approvable by a regulatory agency or listed in a Pharmacopeia or other generally recognized guide for use in animals, and more particularly in humans.

The person skilled in the art will understand that the topical compositions described herein will typically contain a “therapeutically effective amount” or “effective amount” of ingredients. The phrases “therapeutically effective amount” or “effective amount” are used herein to refer to an amount of the active ingredient sufficient to have a therapeutic effect upon administration, e.g. that amount which will cause an improvement or change in the condition for which it is applied when applied to the affected area repeatedly over a period of time. Effective amounts will vary with the particular condition being treated, the severity of the condition, the duration of the treatment, the stage of advancement of the condition, the body surface area affected with the clinical condition, and the specific components of the composition. An effective amount of the active ingredient for treatment of a condition or disorder can be determined by standard clinical techniques. Appropriate amounts in any given instance will be readily apparent to those skilled in the art in light of the present disclosure or capable of determination by routine experimentation. The compositions are generally applied in topical manner to the affected area, i.e. localized application to the skin region where the clinical abnormality is manifest.

### Compositions

The present inventor has determined that a composition comprising one or more magnesium salts, such as magnesium chloride or magnesium sulphate, in combination with one or more organosulfur compounds such as lipoic acid or methylsulfonylmethane (MSM), formulated for topical application can be used to treat and/or prevent viral infections and skin conditions such as herpes zoster infection, varicella-zoster infection, chicken pox, shingles, hidradenitis suppurativa, psoriasis, acne, atopic dermatitis, atopic eczema and postherpetic neuralgia.

Any suitable magnesium salt or salts may be used, for example it may be magnesium chloride, magnesium sulphate, or a mixture thereof. In some embodiments, the composition comprises magnesium sulfate heptahydrate. In some embodiments, the composition comprises magnesium chloride hexahydrate. In some embodiments, the composition comprises both magnesium sulfate heptahydrate and magnesium chloride hexahydrate.

In some embodiments, the composition comprises an amount of magnesium sulfate heptahydrate in the range of from 10 to 65% by weight, or from 45 to 65% by weight.

In some embodiments, the composition comprises an amount of magnesium chloride in the range of from 5 to 35% by weight, or from 20 to 35% by weight.

In some embodiments, the composition comprises an amount of alpha-lipoic acid in the range of from 0.1 to 1% by weight.

In some embodiments, the composition comprises an amount of methylsulfonylmethane in the range of from 10 to 20% by weight. Methylsulfonylmethane is also known as dimethyl sulfone.

In some embodiments, the composition comprises magnesium chloride, alpha-lipoic acid and methylsulfonylmethane (MSM).

In some embodiments, the composition comprises magnesium chloride, magnesium sulfate (e.g. magnesium sulfate heptahydrate), alpha-lipoic acid and methylsulfonylmethane (MSM) In some embodiments, the composition comprises a buffer. In some embodiments, the composition comprises a base.

In some embodiments, the composition comprises a bicarbonate or carbonate, for example sodium bicarbonate.

In some embodiments, the composition comprises lemon extract, for example lemon juice. In some embodiments, the composition comprises from 50% to 80% by weight lemon extract.

In some embodiments, the composition comprises: (a) magnesium chloride and/or magnesium sulphate; and (b) lipoic acid and/or methylsulfonylmethane (MSM), dissolved/suspended in water or other suitable aqueous solvent. In another embodiment, the topical composition further comprises one or more of sodium bicarbonate and lemon extract.

In some embodiments, the composition comprises: (a) magnesium chloride and/or magnesium sulphate; and (b) lipoic acid and/or methylsulfonylmethane, water or other aqueous solvent, and one or more oil-phase components.

In some embodiments, the composition comprises Piper Betle vine leaf oil.

In some embodiments, the composition comprises a surfactant, for example such as polysorbate 20.

In some embodiments, the composition comprises Piper Betle vine leaf oil and polysorbate 20.

In some embodiments, the composition comprises a gum, for example xanthan gum.

In some embodiments the composition comprises by dry weight of the composition: 45%-65% magnesium sulphate, 10%-20% MSM, 20%-35% magnesium chloride, 1%-10% sodium bicarbonate and/or 0.1%-1% alpha-lipoic acid.

In some embodiments, the composition comprises by weight of the composition: 10%-65% magnesium sulphate, 5%-20% MSM, 5%-35% magnesium chloride, 1%-10% sodium bicarbonate and/or 0.1%-1% alpha-lipoic acid.

In some embodiments, the composition comprises 20%-50% by weight of the composition of magnesium chloride, alpha-lipoic acid, sodium bicarbonate, magnesium sulphate, and methylsulfonylmethane (MSM).

In some embodiments, the composition comprises magnesium chloride, magnesium sulfate, methylsulfonylmethane (MSM), alpha-lipoic acid, sodium bicarbonate and xanthan gum.

In some embodiments, the composition comprises 51% w/w magnesium sulfate heptahydrate, 26.2% w/w magnesium chloride hexahydrate, 15.7% w/w methylsulfonyl methane, and 0.5% alpha-lipoic acid.

In some embodiments, the composition comprises 18-22% w/w magnesium sulfate hexahydrate, 8-12% w/w magnesium chloride hexahydrate, 0.5-2% w/w methylsulfonyl methane, and 0.1-0.3% w/w alpha-lipoic acid.

In some embodiments, the composition is provided in the form of an aqueous solution.

In some embodiments, the composition is an aqueous solution containing magnesium chloride, magnesium sulfate, methylsulfonylmethane (MSM), alpha-lipoic acid and sodium bicarbonate.

In some embodiments, the composition is an aqueous solution containing magnesium chloride hexahydrate, magnesium sulfate heptahydrate, methylsulfonylmethane (MSM), alpha-lipoic acid and sodium bicarbonate.

In some embodiments, the composition is a 50 ml aqueous solution containing magnesium chloride hexahydrate (7.85 g), magnesium sulfate heptahydrate (15.31 g), methylsulfonylmethane (MSM) (4.71 g), alpha-lipoic acid (0.16 g) and sodium bicarbonate.

In some embodiments two sachets are supplied containing powders with a total net weight of 30 g. Powder Mix A contains the following ingredients: Magnesium sulfate heptahydrate (15.31 g), Magnesium chloride hexahydrate (7.85 g), Dimethyl sulfone (4.71 g), Alpha lipoic acid (0.16 g). Powder B contains: Sodium bicarbonate. The sachets containing powder are for combining with water to create a 50 mL solution.

In some embodiments, the composition is formulated for topical administration.

The compositions of the present disclosure may for example be formulated by those skilled in the art as liquids, toners, aqueous solutions, sprays, emulsions, moisturizers, sunscreens, creams, lotions, masks, suspensions, triturates, gels, jellies, pastes, foams, ointments, creams, adhesives, serums, treated clothes or pads and the like.

In some embodiments, the compositions may be applied to the skin by any means known in the art including, but not limited to, by an aerosol, spray, pump-pack, brush, swab, or other applicator. Optionally, the applicator provides either a fixed or variable metered dose application such as a metered dose aerosol, a stored-energy metered dose pump or a manual metered dose pump.

In some embodiments, the composition as described herein is an aqueous solution. The pharmaceutical composition may for example comprise water in an amount from about 50% to about 99.9% by weight, or from about 70% to about 99.9% by weight. Suitably, the pH of the composition is adjusted to a pH of between about 2 to about 6, but preferably to about 4 to about 6, such as about 4.5 to about 5.5. The aqueous solution may also comprise one or more of a co-solvent, a humectant, a chelating agent, an antioxidant, a preservative, a fragrance, a colorant or a penetration enhancer as known in the art. The aqueous solution may be applied to skin using any suitable technique, and in one embodiment is applied as a spray.

In some embodiments, the composition as described herein is an aqueous gel. In this embodiment, the pharmaceutical composition comprises water in an amount from about 50% to about 99% by weight, such as from about 70% to about 99% by weight. Suitably, the pH of the composition is adjusted to a pH of between about 2 to about 6, but more particularly to about 4 to about 6, or about 4.5 to about 5.5. For pH adjustment, any agent commonly used in the laboratory for pH adjustment, such as hydrochloric acid or sodium hydroxide, can be used. Other acids such as acetic acid, benzoic acid, formic acid, fumaric acid, lactic acid, phosphoric acid, sulfuric acid or other organic and/or inorganic acids, as known to those skilled in the art, could also be used. Other bases such as ammonium hydroxide, ethanolamine, magnesium hydroxide, sodium or potassium bicarbonate, sodium or potassium hydroxide, organic and/or inorganic bases, as known to those skilled in the art, could also be used. Furthermore, in this embodiment, the pharmaceutical composition will also comprise a suitable gelling agent. The composition may further comprise a co-solvent, a humectant, a chelating agent, an antioxidant, a preservative, a fragrance, a colorant or a penetration enhancer, or a combination or mixture thereof.

In another embodiment, the dermatologically acceptable formulation is a cream. In one embodiment, the cream is an oil-in-water cream. Suitably, the oil-in-water cream comprises an oil phase, a water phase, a surfactant and an antioxidant.

In some embodiments, the composition is formulated as an emulsion. In some embodiments the emulsion comprises an emulsifier, for example glyceryl stearate, ceteareth-20, ceteareth-12, cetearyl alcohol, cetyl palmitate, or mixtures thereof.

In some embodiments the dermatologically acceptable formulation comprises one or more emollients and/or humectants. Emollients and humectants suitable for the composition include glycerol, glyceryl stearate, Ceteareth-20, Ceteareth-12, cetearyl alcohol, cetyl palmitate, dimethicone, cyclopentasiloxane, cyclohexasiloxane, caprylic/capric triglyceride, or mixtures thereof.

In some embodiments the dermatologically acceptable formulation comprises one or more preservatives. Preservatives suitable for the composition include phenoxyethanol, ethylhexylglycerin, sodium benzoate, potassium sorbate, sorbic acid, methylparaben, or mixtures thereof.

In embodiments described herein, viscosifiers are added to the topical compositions to increase the viscosity to enable it to adhere to a surface, or to improve how the composition feels to one's touch when it is being used or applied. In embodiments described herein, a thickening agent is added to the topical composition to stabilize the dispersion until use. In embodiments described herein, viscosifiers utilized include cetearyl alcohol, carageenans, cellulose compounds such as methyl cellulose, hydroxymethyl cellulose, acrylamide/sodium acryloyldimethyl taurate copolymer and carboxymethyl cellulose; pectin; dextrans of various molecular weight ranges; starch; gum tragacanth; gum arabic; guar gum; acacia gum; gum karaya; silica, diatomaceous earth; and other commonly used agents known to those skilled in the art. In embodiments described herein, the general range for the addition of a viscosifier is in the range from about 0.001% to about 10%.

In embodiments described herein, other ingredients such as, but not limited to, preservatives, colorants and fragrances are added to the topical composition. Examples of preservatives include phenoxyethanol, ethylhexylglycerin, butylated hydroxyanisole (“BHA”), butylated hydroxytoluene (“BHT”), phenol, resorcinol; parabens such as methyl paraben, ethyl paraben, propyl paraben, butyl paraben, isopropyl paraben, isobutyl paraben, 2-phenoxyethanol, 1,3-Octandiol; sodium benzoate, benzyl alcohol, or other preservatives commonly used in the industry. Other phenolic agents include 4,6-di-tert-butyl-resorcinol, 2,6-di-tert-butylphenol, 2,5-di-tert-butylphenol, 3,5-di-tert-butylphenol, 2,6-di-tert-hexylphenol, 2,6-di-tert-octylphenol and 2,6-di-tert-decylphenol. The general range for addition of preservatives is from about 0.01% to about 10% by weight of the composition.

In embodiments described herein, fragrances are added to the composition. The fragrance is added to the composition in a range from about 0.001% to about 20%. Examples of fragrances that could be used include, for example only and are not intended to be limited, ammonium glycyrrhizate, amyl acetate, anisaldehyde, benzoic acid, Betula alba extract, caraway fruit oil, safflower seed oil, caramel, cedarwood oil, cinnamyl acetate, citrus extracts such as from orange, grapefruit, lemon or lime, citronella, carrot, clove, eucalyptus, wintergreen, licorice, lavender, cherry, various berries or the like.

In some embodiments, the composition comprises a pharmaceutically and/or dermatologically acceptable excipient comprising an ingredient selected from: polysaccharide polymer, glycerin, glycerol, butylene glycol, dipropylene glycol, pentylene glycol, polyethylene glycol, ethanol, and water. In embodiments described herein, the pharmaceutically and/or dermatologically acceptable excipient may also include one or more cosmetically or pharmaceutically acceptable carriers. Suitable carriers that may be used in the topical compositions discussed herein are known in the art and include, but are not limited to, solubilizers such as C₂ to C₈ straight and branched chain alcohols, diols and triols, moisturizers and humectants such as glycerine, glycerol, amino acids and amino acid derivatives, polyaminoacids and derivatives, pyrrolidone carboxylic acids and their salts and derivatives, surfactants such as sodium laureth sulfate, sorbitan monolaurate, emulsifiers such as cetyl alcohol, stearyl alcohol, glyceryl stearate ceteareth-20, ceteareth-12 cetearyl alcohol, cetyl palmitate, or mixtures thereof, emollients such as dimethicone, cyclopentasiloxane, cyclohexasiloxane, caprylic/capric triglyceride, lanolin, mineral oil, petrolatum, cetyl alcohol, thickeners such as xanthan gum, methyl cellulose, ethyl cellulose, hydroxymethylcellulose, hydroxypropylcellulose, polyvinylpyrrolidone, polyvinyl alcohol and acrylic polymers. Other examples of suitable excipients, such as binders and fillers are listed in Remington's Pharmaceutical Sciences (18th Edition, Ed. Alfonso Gennaro, Mack Publishing Co. Easton, Pa., 1995) and Handbook of Pharmaceutical Excipients (3rd Edition, Ed. Arthur H. Kibbe, American Pharmaceutical Association, Washington, D.C. 2000). In one embodiment, the composition comprises Xanthan gum.

In some embodiments, the compositions may comprise one or more additional active and/or therapeutic agents, for example an active and/or therapeutic agent useful in the treatment of viral and/or skin conditions, for example an agent useful in the treatment of a condition such as herpes zoster infection, varicella-zoster infection, chicken pox, shingles, hidradenitis suppurativa, psoriasis, acne, atopic dermatitis, atopic eczema and/or postherpetic neuralgia.

In some embodiments, the pharmaceutical composition is for administration in combination with a further therapeutic and/or active agent.

In some embodiments, the further therapeutic agent is an antiviral agent, an antibiotic, an antiseptic agent, an antifungal, an antibacterial agent, an analgesic or an anti-inflammatory agent. In additional embodiments, the topical compositions of the invention may further comprise anaesthetics, anti-acne agents, humectants, such as cationic, ionic and non-ionic surfactant, moisturizers, antipruritic agents, antiperspirants, anti-psoriatic agents, anti-seborrheic agents, antiaging and anti-wrinkle agents, skin lightening agents, depigmenting agents and vitamins.

Exemplary antiviral agents include, but are not limited to, penciclovir, acyclovir, cidofovir, idoxuridine, edoxudine, caffeine, trifluorothymidine, nonoxynol-9, glutaraldehyde, povidone-iodine, and ascorbic acid.

Exemplary antibiotics include, but are not limited to, ampicillin, bacampicillin, carbenicillin indanyl, mezlocillin, piperacillin, ticarcillin, amoxicillin-clavulanic acid, ampicillin-sulbactam, benzylpenicillin, cloxacillin, dicloxacillin, methicillin, oxacillin, penicillin G, penicillin V, piperacillin tazobactam, ticarcillin clavulanic acid, nafcillin, procaine penicillin, cefadroxil, cefazolin, cephalexin, cephalothin, cephapirin, cephradine, cefaclor, cefamandol, cefonicid, cefotetan, cefoxitin, cefprozil, ceftmetazole, cefuroxime, loracarbef cefdinir, ceftibuten, cefoperazone, cefixime, cefotaxime, cefpodoxime proxetil, ceftazidime, ceftizoxime, ceftriaxone, cefepime, azithromycin, clarithromycin, clindamycin, dirithromycin, erythromycin, lincomycin, troleandomycin, cinoxacin, ciprofloxacin, enoxacin, gatifloxacin, grepafloxacin, levofloxacin, lomefloxacin, moxifloxacin, nalidixic acid, norfloxacin, ofloxacin, sparfloxacin, trovafloxacin, oxolinic acid, gemifloxacin, perfloxacin, imipenem-cilastatin, meropenem, and aztreonam. In one embodiment, the amount of the antibiotic in the composition is from 0.01 to 5% by weight of the total composition.

Antiseptic compounds include, but are not limited to, iodine, manuka honey, octenidine dihydrochloride, phenol, polyhexanide, sodium chloride, sodium hypochlorite, calcium hypochlorite, sodium bicarbonate, methyl paraben, and sodium dehydroacetate. In one embodiment, the amount of the antiseptic compound in the topical formulation is from 0.01 to 5% by weight of the total composition.

Antifungal agents include, but are not limited to, amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, benzoic acid, ciclopirox, flucytosine, griseofulvin, haloprogin, tolnaftate, undecylenic acid, crystal violet, and balsam of Peru. In one embodiment, the amount of the antifungal agent in the topical formulation is from 0.01 to 5% by weight of the total composition.

Analgesic agents include, but are not limited to, methyl salicylate, codeine, morphine, methadone, pethidine, buprenorphine, hydromorphine, levorphanol, oxycodone, fentanyl, and a non-steroidal anti-inflammatory drug. The amount of the analgesic agent in the topical formulation is from 0.01 to 5% by weight of the total composition.

In some embodiments, the composition does not contain a further active or therapeutic agent.

In some embodiments described herein, the composition is a topical composition. In some embodiments, the composition includes a secondary skin care active. The term “skin care active” refers to a compound or substance that provides a benefit when applied to skin. Skin care actives may provide benefits, or claimed benefits, in areas such as one or more of wrinkle removal or wrinkle reduction, firming of skin, exfoliation of skin, treatment of acne, skin conditioning, improvement of skin barrier properties, control of sweat, anti-aging, reduction or avoidance of irritation and reduction or avoidance of inflammation. Examples of skin care actives include molecules such as peptides, proteins, oligonucleotides, fullerenes as well as small molecules. Skin care actives may be protease and/or enzyme inhibitors, anti-coenzymes, chelating agents, antibodies, antimicrobials, humectants, vitamins, skin protectants, antioxidants and/or skin soothing agents, plant extracts and the like. Examples of skin care actives include but are not limited to vitamin C, vitamin E (alpha tocopherol), retinoids, soy derivatives (e.g. isoflavones), green tea polyphenols, alpha hydroxy acids (e.g. glycolic and lactic acids), beta hydroxy acids (e.g. salicylic acid), poly hydroxy acids, alpha lipoic acid, hemp oil (glycerides), niacinamide, dimethyl amino ethanol, coenzyme Q10, capryloyl glycine, undecylenoyl glycine, octenidine HCl, elubiol, 1,2 hexanediol, oligopeptide-10, bakuchiol, 4-Hydroxy-cyclohexanecarboxylic acid butyl ester, nigella sativa oil, cinnamon bark extract, quercetin, ECGC, blueberry extract, resveratrol and pterostilbene, betaglucan, kinetin (plant growth hormone), dimethyl sulfone and botulinum toxin. Other examples of skin care actives may be found in The Perricone Prescription (Nicholas Perricone, Harper Collins Publishers Inc., New York, 2002).

### Manufacture of the Composition

There is also provided a method of making a pharmaceutical composition as disclosed herein, the method comprising: combining a magnesium salt with methysulfonylmethane (MSM) and/or alpha-lipoic acid to form a mixture; and combining the mixture with water or an aqueous solution.

For example, compositions according to the present disclosure may be prepared as follows: the dry ingredients (e.g., magnesium sulphate, methylsulfonylmethane (MSM), alpha-lipoic acid and sodium bicarbonate) may be mixed together, and then subsequently dissolved/suspended in a suitable aqueous solvent. The aqueous solvent may for example be water, a buffered water solution and/or comprise lemon extract.

In some embodiments, the method comprises: (i) combining magnesium sulphate, methylsulfonylmethane (MSM), alpha-lipoic acid and sodium bicarbonate to form a dry ingredient mixture, (ii) adding magnesium chloride to the dry ingredient mixture, (iii) combining the dry ingredient mixture formed in step (ii) with water or an aqueous solution to form the topical composition.

In some embodiments, the composition comprises by weight of the composition: 10%-65% magnesium sulphate, 5%-20% MSM, 5%-35% magnesium chloride, 1%-10% sodium bicarbonate and/or 0.1%-1% alpha-lipoic acid.

In some embodiments, the mixture formed in step (ii) comprises by dry weight of the composition: 45%-55% magnesium sulphate, 10%-20% MSM, 20%-30% magnesium chloride, 1%-10% sodium bicarbonate and 0.1%-1% alpha-lipoic acid.

In some embodiments, the pharmaceutical composition comprises 20%-50% by weight of the composition of magnesium chloride, alpha-lipoic acid, sodium bicarbonate, magnesium sulphate, and methylsulfonylmethane (MSM), and 50%-80% by weight of water or an aqueous solution.

In some embodiments an aqueous solution is added, and wherein the aqueous solution comprises lemon extract. In some embodiments, the lemon extract is lemon juice.

In some embodiments, the method comprises the step of adding an oil (e.g. Piper Betle vine leaf oil).

In some embodiments, a surfactant (e.g. polysorbate 20) is added.

In some embodiments, a mixture of Piper Betle vine leaf oil and Polysorbate 20 is added. The mixture of Piper Betle vine leaf oil and Polysorbate 20 may be in a weight ratio in the range of from 2:1 to 6:1, 3:1 to 5:1, Piper Betle vine leaf oil to Polysorbate 20. Typically, the ratio of Piper Betle vine leaf oil to Polysorbate 20 is 4:1.

If desired, the composition can be prepared contemporaneously, e.g. just prior to administration. For example, a kit or package containing containers with different components of the composition may be provided. For example, one or more containers containing dry/solid ingredients may be provided, and one or more containers containing liquid/aqueous ingredients may be provided.

In some embodiments, a package or kit comprising at least two containers is provided, for example a first container containing magnesium chloride, alpha-lipoic acid and/or MSM, and a second container containing aqueous solvent (e.g. lemon juice). In some embodiments, a further container is provided, containing sodium bicarbonate and magnesium sulfate. In some embodiments, a further container is provided containing Piper Betle vine leaf oil and polysorbate 20.

Any suitable form of container may be used, for example a pouch, capsule, packet or the like, together with instructions for dissolving/suspending the dry ingredients in a suitable aqueous solvent such as water prior to topical application. The dry ingredients can be combined in a single container, or combinations of ingredients, or single ingredients, provided in separate containers. The patient may also be provided with a container of aqueous solvent, for example buffered sterile water, into which the dry ingredients are mixed.

### Therapeutic Uses

The compositions as described herein can be used for the treatment, prevention and/or reduction of severity of viral and/or skin conditions.

Without being bound by any theory, it is considered that the present compositions have effects in one or more of i) inactivating, stopping and/or retarding the progress of the virus; ii) facilitating the effectiveness of the body's healing processes and/or immune system processes; and iii) alleviating symptoms such as those manifesting on the skin and/or associated with pain, including neuropathic pain.

Examples of conditions which the compositions find use in therapy of include herpes zoster infection, varicella-zoster infection, herpes simplex infection, chicken pox, shingles, cold sores, genital herpes, hidradenitis suppurativa, psoriasis, acne, atopic dermatitis, atopic eczema, postherpetic neuralgia, pulmonary fibrosis and SARS-CoV-2.

Examples of symptoms which the composition finds use in therapy of, include cracked skin, skin bleeding, skin peeling, skin redness and pain, including neuropathic pain.

Examples of conditions which the composition finds use in therapy of, include conditions caused by or associated with the varicella-zoster virus, and/or viral skin conditions. Examples of such conditions include herpes zoster and chickenpox (also known as varicella), and/or neuropathy associated with such conditions. In one embodiment, the condition is herpes zoster. In a further embodiment, the condition is a subset of herpes zoster, such as herpes zoster ophthalmicus.

Occurrence of herpes zoster can be associated with COVID-19. There have been reports of COVID-19 patients suffering from herpes zoster. Further, compositions according to the present disclosure have been found to have virucidal activity against the COVID-19 virus. Accordingly, in some embodiments, the compositions find use in the treatment and/or prevention of SARS-CoV-2, and/or of a skin condition associated with SARS-CoV-2, and/or in the treatment and/or prevention of herpes zoster associated with SARS-CoV-2.

In some embodiments, the compositions find use in the treatment and/or prevention of a herpes simplex infection, or a disease or disorder associated with a herpes simplex infection. In some embodiments, the disease or disorder is a herpes simplex type 1 infection, or a disease or disorder associated with a herpes simplex type 1 infection. In some embodiments, the disease or disorder is a herpes simplex type 2 infection, or a disease or disorder associated with a herpes simplex type 2 infection. In some embodiments, the disease or disorder is cold sores or genital herpes.

The present compositions are useful in treating symptoms of skin conditions including pain, redness, rashes, swelling, lesions, abcesses and pustules. Accordingly, in some embodiments, the compositions are used for the prevention and/or treatment of at least one sign or symptom of a skin condition. In some embodiments, the compositions are used for the prevention and/or treatment of at least one sign or symptom of a skin condition selected from the group consisting of pain, redness, rashes, swelling, lesions, abcesses, and pustules.

To treat and prevent skin conditions, the composition is typically applied topically as needed, for example once per week or more, for example one or more times daily to the afflicted skin. In one embodiment, the composition is applied to the skin one or more times daily for a period of several days to several weeks, and until such time as the signs or symptoms of the skin condition have stabilised (ceased progressing) or improved.

In one embodiment, the composition as described herein is applied topically by spraying onto the skin. The composition may if desired be stored in a refrigerator when not in use, and for example shaken well prior to use. The composition may then be sprayed, or applied by hand, onto the tissue to be treated. Typically, the composition is applied to the skin to be treated once daily, although multiple daily applications of the composition are acceptable and safe. In some embodiments, the topical composition is applied once daily, immediately prior to retiring for the evening.

In some embodiments, the composition is administered by inhalation. For example, the composition may be diluted in warm water and the vapours inhaled.

Whilst the precise amount to be administered will vary depending on the condition being treated and the area affected, where the composition is administered daily, the amount of composition administered may for example be in the range of from 0.5 g per day to 100 g per day, or from 2 g per day to 25 g per day, or about 0.5 g, about 1 g, about 2 g, about 3 g, about 4 g, about 5 g, about 6 g, about 7 g, about g, about 9 g, about 10 g, about 15 g, about 20 g, about 25 g, about 30 g, about 40 g, about 50 g, about 60 g, about 70 g, about 80 g, about 90 g, or about 100 g per day. The amount per day may be administered as a single dose, or across multiple doses (e.g. composition may be administered 2, 3, 4 or more times per day).

As would be understood in the art, the composition may be used in combination with other known therapeutic agents suitable for the treatment of herpes zoster infection, varicella-zoster infection, herpes simplex infection, chicken pox, shingles, cold sores, genital herpes, hidradenitis suppurativa, psoriasis, acne, atopic dermatitis, atopic eczema, postherpetic neuralgia, pulmonary fibrosis and SARS-CoV-2 as considered clinically appropriate.

The invention is hereinafter described by way of the following non-limiting Examples and with reference to the accompanying figures.

### EXAMPLES

### Example 1. A Formulation of the Topical Composition

The present inventor formulated the topical composition for testing in trials for treating severe acne vulgaris, hidradenitis suppurativa and psoriasis. The formula of the composition for testing is provided in Table 1.

TABLE 1
Formulation of the topical composition - 1

| Ingredient | Amount | |
|---|---|---|
| Magnesium sulphate heptahydrate | 500 | g |
| Methylsulfonylmethane | 150 | g |
| Sodium bicarbonate | 50 | g |
| Magnesium chloride | 250 | g |
| alpha lipoic acid | 5 | g |
| Lemon extract juice | 50 | ml |

The chemicals in Table 1, except the magnesium chloride and lemon extract juice, were combined for 3 minutes by vigorous shaking to ensure all the dry ingredients were mixed thoroughly. The magnesium chloride was then added and the composition mixed. A sample of 40 g of the mixed ingredients was then dissolved in 100 ml of the lemon extract juice and shaken for 4 minutes. Application of the topical composition to clean glass left a powder residue on drying.

The topical composition was applied to the skin of a test subject, in particular to the arms, face and neck of the subject, over a period of 2 weeks. Prior to use, the topical composition was shaken well and then sprayed onto the skin. After 2 weeks of treatment, there were no adverse reactions or side effects.

### Example 1A. A Formulation of the Topical Composition

The present inventor formulated a further topical composition for testing in trials according to the formula shown in Table 2.

TABLE 2
Formulation of the topical composition - 2

| Ingredient | Amount (g) |
|---|---|
| Purified water | 392.9 |
| Magnesium sulphate heptahydrate | 191.3 |
| Dimethyl sulfone | 8.9 |
| Alpha lipoic acid | 2.0 |
| Magnesium chloride hexahydrate | 98.2 |
| Glycerol | 20.0 |
| Xanthan gum | 5.0 |
| Sodium bicarbonate | 19.7 |
| Emulgade SE-PF | 50.0 |
| Cetearyl alcohol | 10.0 |
| Xiameter PMX-200 200 CS | 20.0 |
| Xiameter PMX-0345 | 50.0 |
| Caprylic/Capric triglyceride | 50.0 |
| Lanolin | 20.0 |
| Euxyl PE 9010 | 10.0 |
| Ethylhexylglycerin | 2.0 |

Note: The commercial reagents described in Table 2 include the following:

| Trade name (commercial | |
|---|---|
| reagent) | Primary component(s) |
| Emulgade SE-PF | Glyceryl Stearate, Ceteareth-20, |
| | Ceteareth-12, Cetearyl |
| | Alcohol, Cetyl Palmitate |
| Lanette D | Cetearyl Alcohol |
| Xiameter PMX-200 200 CS | Dimethicone |
| Xiameter PMX-0345 | Cyclopentasiloxane, Cyclohexasiloxane |
| Crodamol GTGC | Caprylic/Capric Triglyceride |
| Euxyl PE 9010 | Phenoxyethanol, Ethylhexylglycerin |
| Sensiva SC50 | Ethylhexylglycerin |

To prepare the topical composition according to Table 2, the magnesium salts, dimethyl sulfone and alpha lipoic acid were dissolved in the purified water. The glycerol was added with mixing until uniform. The xanthan gum was added slowly and mixed until well dispersed, where the batch thickens. The sodium bicarbonate was added and mixed until totally dissolved. The aqueous mixture was then heated to 70-75 °C.

In a separate vessel, oil phase ingredients were combined (Emulgade SE-PF to Lanolin inclusive) and heated to 70-75 °C. The heated oil phase components were then added to the heated aqueous phase components with mixing until uniform. Mixing was then continued while allowing the batch to cool. Finally, the Euxyl PE9010 and ethylhexyglycerin were mixed in until the composition was uniform.

### Example 2. Treatment of Hidradenitis Suppurativa

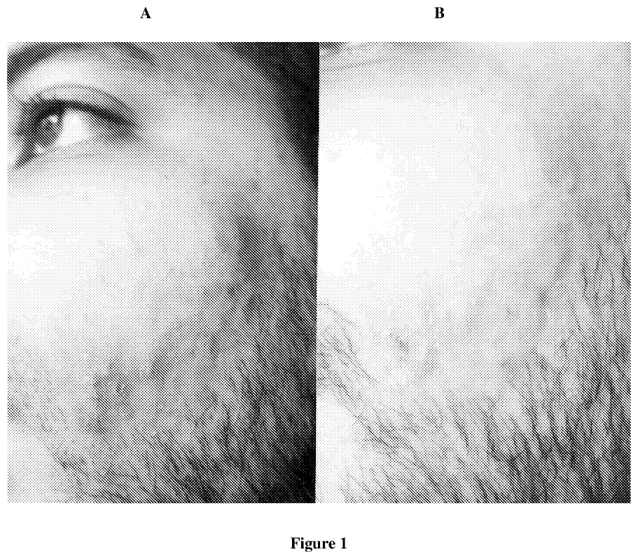

A patient with stage 1 hidradenitis suppurativa (HS) on the face and groin was treated with daily application of the topical composition as described herein for a period of three weeks. The topical composition of Example 1 was applied to the areas of affected skin. After the three week treatment period, the patient reported that the HS had improved significantly. FIG. 1 shows before and after treatment photos of the facial HS. As can be seen in the photographs in FIG. 1, the HS signs and symptoms are significantly reduced after the three week treatment period.

### Example 3. Topical Treatment of Shingles

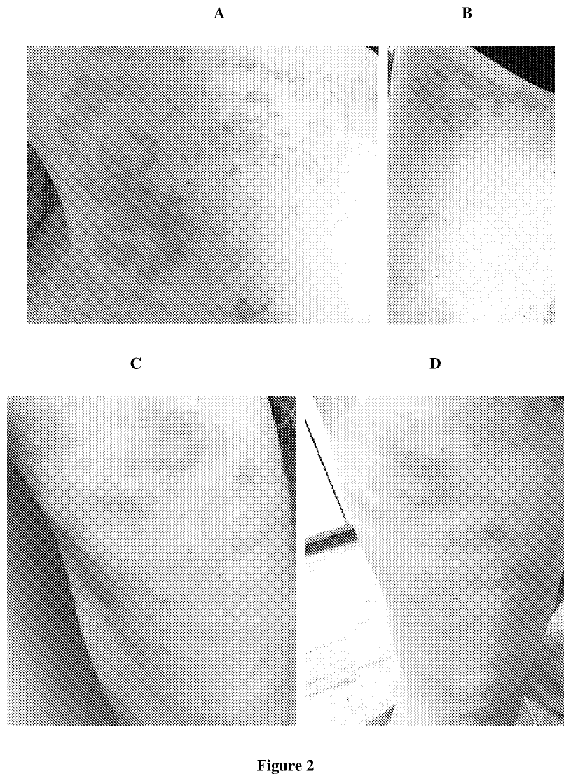

A patient suffering from shingles (herpes zoster), and complaining of significant pain and discomfort was treated with the topical composition as described herein. The composition was applied to the lesions twice daily. Loss of redness and itchiness occurred within a short period following administration. Within a period of a few days signs of viral infection disappeared. Treatment was continued for a 9 day period. FIGS. 2A and B are photographs of the patient's leg showing the extent of the infection and the associated redness and lesions, taken prior to administration of the topical composition. FIGS. 2C and 2D are photographs of the patient's leg following administration of the topical composition twice daily for three days. As can be seen, shingles signs and symptoms were significantly reduced.

### Example 4: Topical Treatment of Shingles

A patient suffering from shingles and having extreme lesions was treated with the topical composition as described herein. The affected areas were the right side of the neck rear and front, right shoulder front and jaw line and ear on the right side. The composition was applied to the head, hair, neck and upper chest area on day 0. After 2 hours the patient reported that the wounds had less pus, and movement was less painful and less restrictive.

The composition was applied twice more at 12 hour intervals, on day 1.

5 days after the third treatment, on day 6, the blistering had completely disappeared, with just scabs remaining.

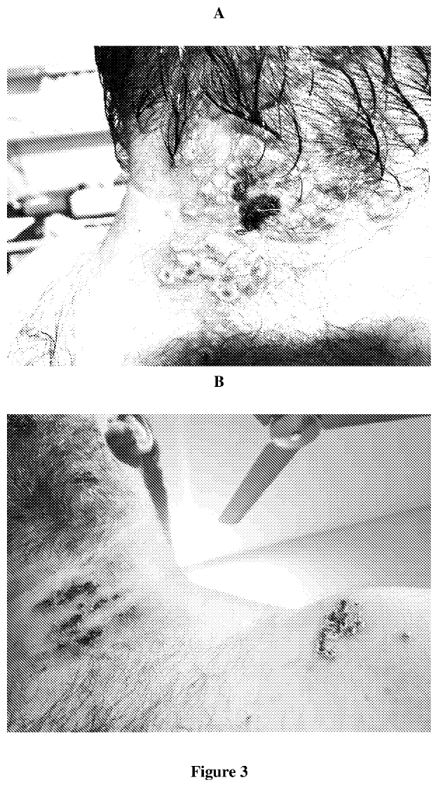

FIG. 3A shows a photograph of an affected area of the patient at the time of receiving the first treatment. FIG. 3B shows a photograph of an affected area of the patient 5 days after the third treatment.

### Example 5. Inhalation Treatment of Shingles—Herpes Zoster Ophthalmicus

A female patient presented suffering from recurrent severe headaches as a complication of shingles. The condition can affect the ophthalmic branch of the trigeminal nerve, leading to hemiplegic migraines and neuropathic pain. The patient had been suffering from the condition for a period of approximately ten years. The patient was administered the composition as defined herein.

The composition was heated in a solution of 50 mL water and 30 mg salts in a porcelain vessel to a temperature that vented vapor, using a microwave. The vessel was partially covered with a towel and the vapor inhaled deeply 10 times through the patient's nose whilst breathing. The procedure was repeated twice daily in the morning and evening for 3 weeks. The patient noted a side effect of a mild ache at the top of the head for 1-2 hours following treatment.

After 3 weeks the patient noted that that no hemiplegic migraine had been experienced over the course of treatment, which was the longest duration the patient had been without a migraine in 10 years. The patient opted to reduce analgesic medication as a result of the decreased instance of headaches. In a follow-up interview conducted four months after treatment, the patient had not undertaken any further treatment and expressed a satisfactory hold on further migraines or neurological symptoms.

### Example 6. Treatment of Postherpetic Neuralgia

A female patient presented suffering from the postherpetic neuralgia complication of shingles. The condition affects nerve fibers and skin, often causing burning pain. The patient had been suffering from the condition for a period of approximately two and a half years. Following administration of the topical composition described herein, the patient reported instant relief from burning soreness. The patient reported that, after a period of time, pain returned but after readministering the composition the burning pain sensation ceased, allowing her to continue with normal activities. In particular, the patient reported that she could now lift and twist her mid-section (an affected area) whilst carrying out manual chores as part of a normal daily routine.

The patient continued administering the composition on an as needed basis over a period of several months, and the patient reported that she no longer experiences burning pain or other symptoms associated with postherpetic neuralgia.

The patient reported that she experienced no side effects.

### Example 7: Treatment of Pulmonary Fibrosis

A patient suffering from pulmonary fibrosis and having difficulty with coughing and reduced mobility due to poor lung capacity for air, was administered the composition as defined herein.

The composition was heated in a solution of 50 mL water and 30 mg salts in a porcelain vessel to a temperature that vented vapor, using a microwave. The vessel was partially covered with a towel and the vapor wafted 28 times into the patients open mouth whilst breathing. The procedure was repeated twice daily for 3 weeks.

After 3 weeks it was noted that coughing rate was greatly diminished, the patient reported that their voice became markedly stronger, and the patient became more mobile which was attributed to increased lung capacity for air. The patient became physically stronger.

### Example 8: Treatment of Idiopathic Pulmonary Fibrosis

A patient suffering from idiopathic pulmonary fibrosis was treated with the composition as defined herein.

The composition was administered via a vapour inhalation process.

The composition was heated in a solution of 50 mL water and 30 mg salts in a vessel to a temperature that vented vapor, using a microwave. The vessel was partially covered with a towel and the vapor wafted times into the patients open mouth whilst breathing. The procedure was repeated twice daily for approximately two months.

The patient improved dramatically over that period, and then stopped taking the treatment. The patient did not consider that he needed its use as he felt well and didn't consider further use was necessary.

The patient went to his clinical doctor in charge of his medicinal case four months later for his annual checkup. The physician was surprised to see that his medical condition had gone into remission, and the patient was now strong enough to join in a possible lung transplant program.

A week later the patient underwent a lung scan at hospital. It was noted that the lungs were stable, reinforcing that the disease had gone into a remission state.

The patient has a steady 120% lung capacity and is hopeful of receiving a transplant in the future should the organs become available. The patient is enjoying a normal life.

### Example 9: Treatment of Atopic Dermatitis

A patient who had suffered from severe atopic dermatitis to the hands for over 20 years, with severe weeping and bleeding of the hands, was treated with the composition described herein. Prior to treatment, the patient had hands which were shredded in the palms leaving large flaps of skin separated from the central palm area. The fingers on each hand had visible open cut sores that were weeping blood.

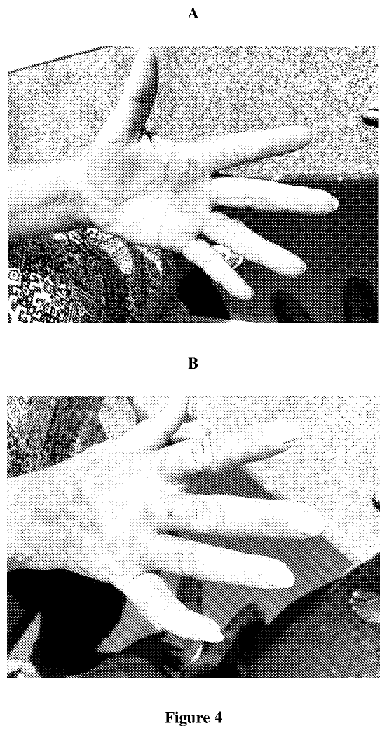

The patient was administered the formulation topically to her hands, The next day, photographs of the hands were taken (see FIG. 4), by which time the angry redness and pain had significantly decreased, weeping had stopped and the fissures were sealing.

Treatment was continued over the following weeks, and the patient showed a complete recovery from atopic dermatitis.

### Example 10: In Vitro Testing—Herpes Simplex Type 1

Samples of a formulation according to the present disclosure as described in Example 1 were tested for virucidal activity against Herpes Simplex Type 1 virus in vitro. Virucidal Test by Carrier Method was conducted using protocol: TMCV 006, ASTM E1053. The test conditions were as follows:

| Conditions | |
|---|---|
| Virus Strain | Herpes Simplex Type 1 ATCC/VR-733 |
| Cell Substrate | Vero cells ATCC/CCL-81 |
| Test Concentration | Neat |
| Contact Time | 30 seconds |
| Test Temperature | Room temperature |
| Test Condition | Clean |
| Neutraliser | 2% FBS in MM |

The virucidal results following 30 seconds contact for various virus dilutions are shown in the table below.

Herpes Simplex Type 1 test/control results for 30 seconds contact

| | Number of | | | | | | |
|---|---|---|---|---|---|---|---|
| Virus | Inoculated | Virus | | | | Test | |
| Dilution | Wells | Control | Percentage | Cytotoxicity | Neutralisation | Sample | Percentage |
| 10⁻¹ | 4 | 4⁺/4 | 100.00 | C | C | C | N/A |
| 10⁻² | 4 | 4⁺/4 | 100.00 | C | C | C | N/A |
| 10⁻³ | 4 | 4⁺/4 | 100.00 | 0⁺/4 | 0⁺/4 | 0⁺/4 | 0.00 |
| 10⁻⁴ | 4 | 4⁺/4 | 100.00 | N/A | N/A | 0⁺/4 | 0.00 |
| 10⁻⁵ | 4 | 4⁺/4 | 100.00 | N/A | N/A | 0⁺/4 | 0.00 |
| 10⁻⁶ | 4 | 4⁺/4 | 100.00 | N/A | N/A | 0⁺/4 | 0.00 |
| 10⁻⁷ | 4 | 4⁺/4 | 100.00 | N/A | N/A | N/A | N/A |
| 10⁻⁸ | 4 | 4⁺/4 | 100.00 | N/A | N/A | N/A | N/A |
| 10⁻⁹ | 4 | 3⁺/4 | 75.00 | N/A | N/A | N/A | N/A |
| Log₁₀ | — | 9.33 | — | 2.5 | 2.5 | 2.5 | — |

| | |
|---|---|
| Log₁₀ Reduction of Virus after Treatment | 6.83 |

Note:
Presence of virus in each response is recorded as “+”
Absence of virus in each response is recorded as “0”
Cytotoxic response is recorded as “C”
Calculated virus titre = 109.83 TCID_{50/0.1ml} (9.33 log₁₀)
Cell control—4 wells with healthy cell monolayer
The Reed & Muench LD50 method was used for determining the virus titre endpoint.

Conclusions: The formulation demonstrated virucidal activity against Herpes Simplex Type 1 by achieving a 6.83 log reduction in virus concentration after 30 seconds exposure period at room temperature.

### Example 11: In Vitro Testing—Herpes Simplex Type 2

Samples of a formulation according to the present disclosure as described in Example 1 were tested for virucidal activity against Herpes Simplex Type 2 virus in vitro. Virucidal Test by Carrier Method was conducted using protocol: TMCV 006, ASTM E1053. The test conditions were as follows:

| Conditions | |
|---|---|
| Virus Strain | Herpes Simplex Type 2 ATCC/VR-734 |
| Cell Substrate | Vero cells ATCC/CCL-81 |
| Test Concentration | Neat |
| Contact Time | 30 seconds |
| Test Temperature | Room temperature |
| Test Condition | Clean |
| Neutraliser | 2% FBS in MM |

The virucidal results following 30 seconds contact for various virus dilutions are shown in the table below.

Herpes Simplex Type 1 test/control results for 30 seconds contact

| | Number of | | | | | | |
|---|---|---|---|---|---|---|---|
| Virus | Inoculated | Virus | | | | Test | |
| Dilution | Wells | Control | Percentage | Cytotoxicity | Neutralisation | Sample | Percentage |
| 10⁻¹ | 4 | 4⁺/4 | 100.00 | C | C | C | N/A |
| 10⁻² | 4 | 4⁺/4 | 100.00 | C | C | C | N/A |
| 10⁻³ | 4 | 4⁺/4 | 100.00 | 0⁺/4 | 4⁺/4 | 0⁺/4 | 0.00 |
| 10⁻⁴ | 4 | 4⁺/4 | 100.00 | N/A | N/A | 0⁺/4 | 0.00 |
| 10⁻⁵ | 4 | 4⁺/4 | 100.00 | N/A | N/A | 0⁺/4 | 0.00 |
| 10⁻⁶ | 4 | 4⁺/4 | 100.00 | N/A | N/A | 0⁺/4 | 0.00 |
| 10⁻⁷ | 1 | 3⁺/4 | 85.71 | N/A | N/A | N/A | N/A |
| 10⁻⁸ | 4 | 2⁺/4 | 50.00 | N/A | N/A | N/A | N/A |
| 10⁻⁹ | 4 | 1⁺/4 | 14.29 | N/A | N/A | N/A | N/A |
| Log₁₀ | — | 7.5 | — | 2.5 | 2.5 | 2.5 | — |

| | |
|---|---|
| Log₁₀ Reduction of Virus after Treatment | 5 |

Note:
Presence of virus in each response is recorded as “+”
Absence of virus in each response is recorded as “0”
Cytotoxic response is recorded as ″C″
Calculated virus titre = 107.5 TCID_{50/0.1ml} (7.5 log₁₀)
Cell control—4 wells with healthy cell monolayer
The Reed & Muench LD50 method was used for determining the virus titre endpoint.

Conclusions: The formulation demonstrated virucidal activity against Herpes Simplex Type 1 by achieving a 5.0 log reduction in virus concentration after 30 seconds exposure period at room temperature.

### Example 10. In Vitro Testing—Murine Hepatitis Virus (MHV1)

Samples of a formulation according to the present disclosure as described in Example 1 were tested for virucidal activity against Murine hepatitis virus (MHV1) in vitro. Virucidal Test by Carrier Method was conducted using protocol: TMCV 006, ASTM E1053. The test conditions were as follows:

| Conditions | |
|---|---|
| Virus Strain | Murine hepatitis virus |
| | (MHV1) ATCC/VR-261 |
| Cell Substrate | A9 cells ATCC/CCL-1.4 |
| Test Concentration | Neat |
| Contact Time | 30 seconds |
| Test Temperature | Room temperature |
| Test Condition | Clean |
| Neutraliser | 2% FBS in MM |

The virucidal results following 30 seconds contact for various virus dilutions are shown in the table below.

Murine Hepatitis Virus (MHV1) test/control results for 30 seconds contact

| | Number of | | | | | | |
|---|---|---|---|---|---|---|---|
| Virus | Inoculated | Virus | | | | Test | |
| Dilution | Wells | Control | Percentage | Cytotoxicity | Neutralisation | Sample | Percentage |
| 10⁻¹ | 4 | 4⁺/4 | 100.00 | C | C | C | N/A |
| 10⁻² | 4 | 4⁺/4 | 100.00 | C | C | C | N/A |
| 10⁻³ | 4 | 4⁺/4 | 100.00 | 0⁺/4 | 4⁺/4 | 4⁺/4 | 100.00 |
| 10⁻⁴ | 4 | 4⁺/4 | 100.00 | N/A | N/A | 1⁺/4 | 25.00 |
| 10⁻⁵ | 4 | 4⁺/4 | 100.00 | N/A | N/A | 0⁺/4 | 0.00 |
| 10⁻⁶ | 4 | 4⁺/4 | 100.00 | N/A | N/A | 0⁺/4 | 0.00 |
| 10⁻⁷ | 4 | 3⁺/4 | 85.71 | N/A | N/A | N/A | N/A |
| 10⁻⁸ | 4 | 2⁺/4 | 50.00 | N/A | N/A | N/A | N/A |
| 10⁻⁹ | 4 | 1⁺/4 | 14.29 | N/A | N/A | N/A | N/A |
| Log₁₀ | — | 7.5 | — | 2.5 | 2.5 | 3.67 | — |

| | |
|---|---|
| Log₁₀ Reduction of Virus after Treatment | 3.83 |

Note:
Presence of virus in each response is recorded as “+”
Absence of virus in each response is recorded as “0”
Cytotoxic response is recorded as ″C″
Calculated virus titre = 107.5 TCID_{50/0.1ml} (7.5 log₁₀)
Cell control—4 wells with healthy cell monolayer
The Reed & Muench LD50 method was used for determining the virus titre endpoint.

Conclusions: The formulation demonstrated virucidal activity against Murine hepatitis virus (MHV1) by achieving a 3.83 log reduction in virus concentration after 30 seconds exposure period at room temperature.

### Example 12: Clinical Study to Evaluate Safety and Efficacy of Topical Composition in Patients with Mild to Moderate Hidradenitis Suppurativa

A study to evaluate the safety and efficacy of topical composition as described herein in patients with mild to moderate hidradenitis suppurativa (as defined by Hurley Stage I or II) was carried out over an 8-week treatment period.

### Objectives:

### Primary:

To evaluate the safety and efficacy of the composition solution when used twice daily in the treatment of mild to moderate hidradenitis suppurativa (as defined by Hurley Stage I or II) over an 8-week treatment period.

### Secondary:

To evaluate the efficacy and effect on dermatological quality of life in participants who apply the composition twice daily in the treatment of mild to moderate hidradenitis suppurativa (as defined by Hurley Stage I or II) over an 8-week treatment period.

### Methodology:

This was an open-label pilot study in which a total of up to 20 participants with mild to moderate hidradenitis suppurativa (Hurley Stage I or II) were to be enrolled. All participants enrolled in the study were to receive active treatment of the composition.

Participants could continue using their regular medications including non-topical HS treatments during the study, however any topical treatment(s) or washes were to be discontinued prior to commencing the study treatment and not to be recommenced until after Week 8 or termination of the study. There was no washout period for the cessation of topical treatment(s).

The study product, was to be applied to all HS lesions twice daily for a duration of 8-weeks. Participants were to cease treatment at Week 8 and were to be followed-up at the Week 10 visit.

Participant visits to the clinic were to be scheduled at Screening/Baseline, Week 4, Week 8 and Week 10.

Participants were also to be followed up with a phone call at Day 7 (±3) after the Screening/Baseline visit.

Clinical determinations of efficacy were to be conducted and assessed by the investigator based on clinical examination and comparison of photos taken at the previous visit. In addition, participants were to complete a daily diary at home and questionnaires at all clinic visits.

Application site reactions, such as dryness, burning and pain, were to be determined at the Screening/Baseline visit and at all subsequent visits.

### Participants

Actual: 19 participants in total were screened and enrolled into the study.
Analysed: 19 participants were included in the Full Analysis Set and 13 participants were included in the Per Protocol population.

### Diagnosis and Main Criteria for Inclusion:

Participants were to be males or females 18 years or older with mild to moderate hidradenitis suppurativa (Hurley Stage I or II), with at least one lesion in at least one distinct anatomical location.

### Test Product, Dose and Mode of Administration:

The composition is supplied as a powder in 2 sachets with a total net weight of 30 g.

### Powder Mix a Contains the Following Active Ingredients:

Magnesium sulfate heptahydrate (15.31 g)
Magnesium chloride hexahydrate (7.85 g)
Dimethyl sulfone (4.71 g)
Alpha lipoic acid (0.16 g)

### Powder B Contains the Inactive Ingredient:

Sodium bicarbonate

Bottled water is added to the combined powders to create a 50 mL solution, which is stored at room temperature (<30 °C) and can be used for up to 7 days.

A small amount of solution was to be applied to all HS lesions in the morning and again prior to going to bed. Participants were to allow the solution to dry on each HS lesion and encouraged not to wash it off.

### Duration of Treatment:

Treatment was to occur for 8 weeks with a 2-week follow-up period.

### Criteria for EVALUATION:

### Safety:

### Safety was Measured Using the Following Primary Endpoint:

Incidence of treatment emergent adverse events (AEs) and serious AEs (SAEs)

### Efficacy:

### Efficacy was Measured Using the Following Primary Endpoint:

Percentage of participants with improvement in the HS Physician Global Assessment (PGA).

### Efficacy was Additionally Measured Using the Following Secondary Endpoints:

Change from baseline in participant's modified Sartorius Score at Weeks 4, 8 and 10.
Change from baseline in participant's total abscess and inflammatory nodule count at Weeks 4, 8 and 10.
Percentage of participants achieving clinical response as measured by HS Clinical Response (HiSCR) at Weeks 4, 8 and 10. Clinical response is defined as at least a 50% reduction from baseline in the total abscess and inflammatory nodule (AN) count, with no increase from baseline in total abscesses.
Proportion of participants who experience an improvement in the Dermatology Life Quality Index (DLQI) from Baseline to Weeks 4, 8 and 10.
Mean change from baseline in the Visual Analogue Scale (VAS) pain scores at Weeks 4, 8 and 10.
Percentage of participants who experience an improvement from Baseline in their Hospital Anxiety and Depression Scale at Weeks 4, 8 and 10.

### SUMMARY—CONCLUSIONS

Nineteen (19) potential participants were screened, and all 19 participants were enrolled into the study and included in the FAS population. Thirteen (13) participants were included in the PP population, with 6 participants excluded as they did not have Week 8 results. Study participants ranged in age from 18 to 43 years, with a mean of 30.9 years. There were 3 (15.8%) males and 16 (84.2%) females. Most participants were Caucasian (n=17, 89.5%). In terms of Hurley clinical staging, 9 (47.4%) participants had Stage I HS and 10 (52.6%) participants had Stage II HS.

### Safety Results:

A total of 13 out of 19 (68.4%) participants experienced 49 adverse events (AEs). All AEs were treatment emergent adverse events (TEAEs). The majority of AEs were mild (35 AEs) or moderate (12 AEs), and only 2 AEs were severe. The numbers of participants experiencing at least one mild, moderate, or severe AE were 12 (63.2%), 5 (26.3%), and 1 (5.3%), respectively. There were no life-threatening or fatal AEs. There was a single serious adverse event (SAE), which was considered unrelated to the IP. There were no withdrawals due to IP.

A total of 5 out of 19 (26.3%) participants experienced 7 mild AEs that were considered at least possibly related to the IP. The preferred term (PT) for these adverse events were application site pain (n=2), application site pruritus (n=2), application site irritation (n=1), pruritus (n=1), and rash (n=1).

Overall, the composition was considered safe and well-tolerated.

### Efficacy Results:

In terms of the primary efficacy endpoint, a total of 6 out of 13 (46.2%) participants had an improved HS-PGA score at Week 4, 5 (38.5%) participants had an improved HS-PGA score at Week 8, and 6 (46.2%) participants had an improved HS-PGA score at Week 10 compared to baseline.

In terms of the secondary efficacy endpoints:
7 out of 13 (53.8%) participants demonstrated a decreased (improved) HS modified Sartorius score (HSMSS) at Week 4, 8 (61.5%) participants demonstrated an improved HS-MSS at Week 8, and 7 (53.8%) participants demonstrated an improved HS-MSS at Week 10 compared to baseline.
6 out of 13 (46.2%) participants demonstrated an improved total abscess and inflammatory nodule (AN) count at Week 4, Week 8 and Week 10 compared to baseline, although the individuals were not the same across the different weeks.
6 out of 13 (46.2%) participants had a HS clinical response (HiSCR) at Week 4, 5 (38.5%) participants had a HiSCR at Week 8, and 6 (46.2%) participants had a HiSCR at Week 10.
9 out of 13 (69.2%) participants experienced an improvement in their Dermatology Life Quality Index (DLQI) score at Week 4, Week 8, and Week 10 compared to baseline. Notably, participants who did not have improvements in their HS-PGA, HS-MSS or total AN count still improved their DLQI score.
Examining the mean change in Overall VAS scores for pain at Weeks 4, 8 and 10 compared to baseline, there was a decrease (i.e. improvement) at each of the visits: by a mean of 7.3 mm (SD=28.12) at Week 4, by a mean of 4.0 mm (SD=24.54) at Week 8 and by a mean of 6.4 mm (SD=15.79) at Week 10. A total of 9 out of 13 (69.2%) participants experienced an improvement in the VAS score at Week 4, 6 (46.2%) participants experienced an improvement in the VAS score at Week 8, and 8 (61.5%) participants experienced an improvement in the VAS score at Week 10 compared to baseline.
The HADS anxiety score showed an improvement in 3 out of 13 (23.1%) participants at Week 4 and an improvement in 9 (69.2%) participants at both Week 8 and Week 10 compared to baseline.
The HADS depression score showed an improvement in 4 out of 13 (30.8%) participants at Week 4 and an improvement in 6 (46.2%) participants at both Week 8 and Week 10 compared to baseline.

### CONCLUSION

When examining the response in individuals, a total of 6 out of 13 (46.2%) participants at Week 4, 5 (38.5%) participants at Week 8, and 6 (46.2%) participants at Week 10 demonstrated an improved HS-PGA compared to baseline. A further 7 out of 13 (53.8%) participants had no change in the HS-PGA score at Week 4, 7 (53.8%) participants had no change in the HS-PGA score at Week 8, and 6 (46.2%) participants had no change in the HS-PGA score at Week 10 compared to baseline. No participants had a worse HS-PGA score at Week 4, and only 1 (7.7%) participant had a worse HS-PGA score at Week 8 and at Week 10.

The participants who demonstrated an improved HS-PGA compared to baseline generally had consistent improvement across multiple efficacy variables, including a decrease in the total AN count from baseline, an overall HS Clinical Response (HiSCR) and an improved HS-MSS. Most of these participants also showed an improved DLQI score and VAS score at the corresponding timepoints compared to baseline. None of the participants recommenced topical HS medications between Week 8 and Week 10, so participants who showed improved measurements at Week 10 compared to baseline were able to improve despite being off IP and other topical HS treatment for two weeks.

The overall safety and tolerability of Hidracare and the promising primary and secondary efficacy results support further research of Hidracare, including a larger Phase II study.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

1. A pharmaceutical composition comprising
(a) a magnesium salt; and
(b) lipoic acid and/or methylsulfonylmethane (MSM)

2. The pharmaceutical composition of claim 1, wherein the magnesium salt is selected from the group consisting of magnesium chloride and magnesium sulphate.

3. The pharmaceutical composition of claim 1, wherein the composition is formulated for topical administration.

4. The pharmaceutical composition of claim 1, wherein the composition comprises magnesium chloride, alpha-lipoic acid and methylsulfonylmethane (MSM).

5. The pharmaceutical composition of claim 1, wherein the composition further comprises sodium bicarbonate.

6. The pharmaceutical composition of claim 1, wherein the composition is in the form of a liquid, an aqueous solution, a gel, a suspension, an emulsion, an ointment or a cream.

7. The pharmaceutical composition of claim 1, wherein the composition comprises by weight of the composition: 10%-65% magnesium sulphate, 5%-20% MSM, 5%-35% magnesium chloride, 1%-10% sodium bicarbonate and/or 0.1%-1% alpha-lipoic acid.

8. The pharmaceutical composition of claim 1, wherein the composition comprises 20%-50% by weight of the composition of magnesium chloride, alpha-lipoic acid, sodium bicarbonate, magnesium sulphate, and methylsulfonylmethane (MSM).

9. The pharmaceutical composition of claim 1, wherein the composition comprises lemon extract.

10. (canceled)

11. The pharmaceutical composition of claim 1, wherein the composition comprises an emollient, humectant, emulsifier, or preservative, or mixtures thereof.

12. The pharmaceutical composition of claim 11, wherein
i) the emollient is selected from the group consisting of glyceryl stearate, ceteareth-20, ceteareth-12, cetearyl alcohol, cetyl palmitate, dimethicone, cyclopentasiloxane, cyclohexasiloxane, and caprylic/capric triglyceride, or a mixture thereof;
ii) the humectant is selected from the group consisting of glycerol, glycerin, salicylic acid, and sorbitol, or a mixture thereof;
ii) the emulsifier is selected from the group consisting of glyceryl stearate, ceteareth-20, ceteareth-12, cetearyl alcohol, and cetyl palmitate, or a mixture thereof; and
iii) the preservative is selected from the group consisting of phenoxyethanol, ethylhexylglycerin, and sodium benzoate, or a mixture thereof.

13. A method of treating, preventing, or reducing the severity of a viral infection, or of a disease or disorder associated with a viral infection, or of a symptom of a viral infection, or of a symptom of a disease or disorder associated with a viral infection, in a subject, comprising administering an effective amount of a pharmaceutical composition according to claim 1 to the subject.

14. The method according to claim 13, wherein the viral infection, or disease or disorder associated with a viral infection, is selected from the group consisting of herpes zoster infection, varicella-zoster infection, herpes simplex infection, chicken pox, shingles, cold sores, genital herpes, hidradenitis suppurativa, psoriasis, acne, atopic dermatitis, atopic eczema, postherpetic neuralgia, pulmonary fibrosis and SARS-CoV-2.

15. The method according to claim 13, wherein the symptom is selected from the group consisting of cracked skin, skin bleeding, skin peeling, skin redness and pain.

16. A method of treating, preventing, or reducing the severity of a disease or disorder, or of a symptom of a disease or disorder in a subject, wherein the disease or disorder is selected from the group consisting of herpes zoster infection, varicella-zoster infection, herpes simplex infection, chicken pox, shingles, cold sores, genital herpes, hidradenitis suppurativa, psoriasis, acne, atopic dermatitis, atopic eczema, postherpetic neuralgia, pulmonary fibrosis and SARS-CoV-2, comprising administering an effective amount of a pharmaceutical composition according to claim 1 to the subject.

17. The method according to claim 13, wherein the composition is administered topically to the subject.

18. The method according to claim 13, wherein the composition is applied to the skin of the subject once daily, twice daily, or three times daily.

19. The method according to claim 18, wherein the topical composition is applied once per day.

20. The method according to claim 13, wherein the composition is applied as a spray, cream or liquid.

21. The method according to claim 13, wherein the composition is administered to the subject by inhalation.

22-54. (canceled)
